(19) United States
(12) Patent Application Publication
(10) Pub. No.: US 2025/0114151 A1
(43) Pub. Date: Apr. 10, 2025

(54) ROTATING MARKER

(71) Applicant: AUGMEDICS LTD., Yokneam Illit (IL)

(72) Inventors: Tomer Gera, Kfar Tavor (IL); Nissan Elimelech, Beerotaim (IL); Nitzan Krasney, Haifa (IL); Stuart Wolf, Yokneam (IL)

(21) Appl. No.: 18/959,270

(22) Filed: Nov. 25, 2024

Related U.S. Application Data
(63) Continuation of application No. 18/008,980, filed on Dec. 8, 2022, now Pat. No. 12,186,028.
Application No. 18/008,980 is a continuation-in-part of application No. 16/901,026, filed on Jun. 15, 2020, now Pat. No. 11,389,252.

(51) Int. Cl. A61B 34/20; A61B 34/10; A61B 90/00; G06T 19/00

(52) U.S. Cl. CPC A61B 34/20; A61B 34/10; A61B 90/39; G06T 19/003; G06T 19/006; A61B 2034/107; A61B 2034/2055; A61B 2090/3904; G06T 2210/41

(57) ABSTRACT

A marker (40) for image guided surgery, consisting of a base (94), having a base axis, connecting to a clamp (42), and an alignment target (44). The alignment target includes a target region (120) having an alignment pattern formed thereon, and a socket (124) connected to the target region and configured to fit rotatably to the base, whereby the alignment target is rotatable about the base axis. The alignment target also includes an optical indicator (162) for the socket indicating an angle of orientation of the alignment target about the base axis.

### CROSS-REFERENCE TO RELATED APPLICATIONS

This application is a continuation of U.S. application Ser. No. 18/008,980, filed Dec. 8, 2022, which is a national stage entry of International PCT Application No. PCT/IB2021/055242, filed Jun. 15, 2021, which is a continuation-in-part of U.S. application Ser. No. 16/901,026, filed Jun. 15, 2020. Each of the foregoing applications is hereby incorporated by reference herein in its entirety.

### FIELD OF THE INVENTION

The present invention relates generally to surgery, and specifically to surgery performed using augmented reality.

### BACKGROUND OF THE INVENTION

In an augmented reality system used by a physician performing surgery, it is typically necessary to register a frame of reference of a patient with a frame of reference of the augmented reality system used by the physician. Methods for registration are known in the art.

U.S. Pat. No. 8,848,977 to Bammer et al., describes a method for optical pose detection. A self-encoded marker where each feature on the pattern is augmented with a 2-D barcode is provided.

U.S. Pat. No. 9,220,573 to Kendrick et al., describes a system for tracking a tracking device for use with a surgical navigation system. The system can include at least one tracking device having a plurality of faces, and the faces can be operable to generate a signal upon activation.

U.S. Pat. No. 9,378,558 to Kajiwara et al., describes a self-position/self-orientation calculation unit calculating self-position and/or self-orientation in a predetermined coordinate system based on a marker in acquired imaged image data when it is determined that the marker exists within a predetermined area.

U.S. Pat. No. 9,495,585 to Bicer et al., describes methods to find one to one mapping between fiducial markers on a tracked object and fiducial marker projections on an image plane captured by a camera in optical object tracking systems.

U.S. Pat. No. 9,943,374 to Merritt et al., describes an image guidance system for tracking a surgical instrument during a surgical procedure. The image guidance system includes a plurality of cameras adapted to be located external to a surgical area for capturing images of optically visible patterns.

U.S. Pat. No. 10,022,104 to Sell et al., describes a marker that includes a first marker component having a first hydrogen proton density and a first mass density; and a second marker component having a second hydrogen proton density different than the first hydrogen proton density.

U.S. Pat. No. 10,080,616 to Wilkinson et al., describes a system which generates a three-dimensional representation of a bone and reference markers, defines a coordinate system for the three-dimensional representation, and determines locations of the reference markers relative to the coordinate system.

U.S. Pat. No. 10,108,833 to Hong et al., describes a marker with a pattern formed thereon, and which includes an optical system. At least a part of the pattern that uniquely appears depending on a direction in which the pattern is viewed from outside of the marker, through the optical system, is visually identified from the outside of the marker.

U.S. Pat. No. 10,251,724 to McLachlin et al., describes a reference tie that may be secured around a portion of a spine during a surgical procedure and that may be tracked by a surgical navigation system.

U.S. Pat. No. 10,296,805 to Yang et al., describes a marker wherein at least one of a position and pose with respect to a capturing unit is estimated.

U.S. Pat. No. 10,420,626 to Tokuda et al., describes methods for automated detection and registration of medical images using fiducial markers and processing algorithms.

U.S. Pat. No. 10,463,434 to Siegler et al., describes tracking marker support structures that include one or more fiducial reference markers, where the tracking marker support structures are configured to be removably and securely attached to a skeletal region of a patient.

U.S. Pat. No. 10,504,231 to Fiala describes fiducial markers that are printed patterns detected by algorithms in imagery from image sensors for applications such as automated processes and augmented reality graphics.

U.S. Pat. No. 10,537,395 to Perez describes a kinematic connector assembly for kinematically coupling two objects. The kinematic connector assembly comprises a receiver defining a cavity and having a plurality of constraint surfaces accessible in the cavity.

U.S. Patent Application 2003/0210812 to Khamene et al., describes an apparatus for pose determination using single camera tracking in a workspace The apparatus includes a computer programmed for making the pose determination and a tracker camera coupled to the computer for providing a tracking image and for which calibration information is stored.

U.S. Patent Application 2011/0098553 to Robbins et al., describes automatic registration of a magnetic resonance (MR) image that is carried out in an image guidance system by placing MR visible markers at known positions relative to markers visible in a camera tracking system.

U.S. Patent Application 2013/0106833 to Fun describes an input device for providing three-dimensional, six-degrees-of-freedom data input to a computer. The device includes a tracker having tracking points. One array of tracking points defines a first axis. Another array defines a second axis or plane orthogonal to the first axis.

U.S. Patent Application 2015/0150641 to Daon et al., describes a three-dimensional position and orientation tracking system that comprises one or more pattern tags, each comprising a plurality of contrasting portions, and a tracker for obtaining image information about the pattern tags.

U.S. Patent Application 2016/0324583 to Kheradpir et al., describes a patient reference device that includes a housing having a back side and a front side, and at least three tracking markers attached to the front side of the housing. The housing extends around the at least three tracking markers and beyond a horizontal plane defined by tops of the at least three tracking markers.

U.S. patent application No. 20170239015 to Sela et al., describes an apparatus that is at least partially visible by both a three dimensional (3D) scanner system of a medical navigation system and a tracking system of the medical navigation system.

Documents incorporated by reference in the present patent application are to be considered an integral part of the application except that, to the extent that any terms are defined in these incorporated documents in a manner that conflicts with definitions made explicitly or implicitly in the present specification, only the definitions in the present specification should be considered.

### SUMMARY OF THE INVENTION

An embodiment of the present invention provides a marker for image guided surgery, including:
a base, having a base axis, connecting to a clamp; and
an alignment target, including:
   a target region having an alignment pattern formed thereon;
   a socket connected to the target region and configured to fit rotatably to the base, whereby the alignment target is rotatable about the base axis; and
   an optical indicator for the socket indicating an angle of orientation of the alignment target about the base axis.

In a disclosed embodiment the socket is configured to only fit to the base in a plurality of at least two discrete orientations about the base axis. Typically, the plurality of discrete configurations is distributed symmetrically about the base axis. The plurality may consist of four discrete orientations.

In a further disclosed embodiment the socket consists of a plurality of apertures equal to the plurality of discrete orientations, and the optical indicator is configured to be visible through one of the apertures indicative of one of the discrete orientations.

In a yet further disclosed embodiment the socket consists of a plurality of apertures equal to the plurality of discrete orientations, and the optical indicator is configured to be visible through apertures selected and arranged so as to provide an unambiguous identification of each of the discrete orientations.

In an alternative embodiment the socket is configured to fit to the base in a plurality of non-discrete orientations about the base axis. The socket may include an aperture, and the optical indicator may be congruent with the aperture, and a fraction of the optical indicator visible through the aperture may be indicative of one of the non-discrete orientations. The aperture may consist of a semicircular arc.

In a further alternative embodiment the socket is at a fixed distance from the target region, and the marker further includes:
an augmented reality system operative during surgery on a patient; and
a processor configured to:
track the alignment target during the surgery,
provide a patient tracking vector to the augmented reality system in response to the tracking of the alignment target,
calculate a change in the angle of orientation of the alignment target in response to changes in images of the optical indicator, and
add a change-of-orientation vector, based only on the fixed distance and the change in the angle of orientation, to the patient tracking vector so as to update the patient tracking vector.

An embodiment of the present invention also provides a method for enabling rotation of a marker during surgery without requiring re-registration, including:
connecting a base, having a base axis, to a clamp;
forming an alignment pattern on a target region of an alignment target;
connecting a socket to the target region, the socket being at a fixed distance from the target region and being configured to fit rotatably to the base, whereby the alignment target is rotatable about the base axis;
providing an optical indicator for the socket indicating an angle of orientation of the alignment target about the base axis;
operating an augmented reality system during the surgery on a patient;
tracking the alignment target during the surgery;
providing a patient tracking vector to the augmented reality system in response to the tracking of the alignment target;
calculating a change in the angle of orientation of the alignment target in response to changes in images of the optical indicator; and
adding a change-of-orientation vector, based only on the fixed distance and the change in the angle of orientation, to the patient tracking vector so as to update the patient tracking vector.

In accordance with aspects of the present disclosure, a marker for image guided surgery is disclosed, the marker includes a base, having a base axis, connecting to an anchoring device; and an alignment target, including: a target region having an alignment pattern formed thereon; a socket connected to the target region and configured to fit rotatably to the base, whereby the alignment target is rotatable about the base axis; and an optical indicator for the socket indicating an angle of orientation of the alignment target about the base axis.

In various embodiments of the marker, the socket is configured to only fit to the base in a plurality of at least two discrete orientations about the base axis.

In various embodiments of the marker, the plurality of discrete configurations is distributed symmetrically about the base axis.

In various embodiments of the marker, the plurality comprises four discrete orientations.

In various embodiments of the marker, the socket comprises a plurality of apertures equal to the plurality of discrete orientations, and the optical indicator is configured to be visible through one of the apertures indicative of one of the discrete orientations.

In various embodiments of the marker, the socket comprises a plurality of apertures equal to the plurality of discrete orientations, and the optical indicator is configured to be visible through apertures selected and arranged so as to provide an unambiguous identification of each of the discrete orientations.

In various embodiments of the marker, the socket is configured to fit to the base in a plurality of non-discrete orientations about the base axis.

In various embodiments of the marker, the socket comprises an aperture, and the optical indicator is congruent with the aperture, and a fraction of the optical indicator visible through the aperture is indicative of one of the non-discrete orientations.

In various embodiments of the marker, the aperture comprises a semicircular arc.

In accordance with aspects of the present disclosure, an augmented reality system or a navigation and/or tracking system operative during surgery on a patient is provided, the system includes the marker, wherein the socket is at a fixed distance from the target region; and a processor configured to or a storage medium storing machine-executable instructions configured to execute on a computing system the instructions, when executed, cause the computing system to: track the alignment target during the surgery, provide a patient tracking vector in response to the tracking of the alignment target, calculate a change in the angle of orientation of the alignment target in response to changes in images of the optical indicator, and add a change-of-orientation vector, based only on the fixed distance and the change in the angle of orientation, to the patient tracking vector so as to update the patient tracking vector.

In various embodiments of the marker, the marker further includes the anchoring device.

In various embodiments of the marker, the anchoring device is a clamp or a pin.

In accordance with aspects of the present disclosure, a marker for image guided surgery is provided, the marker includes: a base, having a base axis; an interface configured to be coupled to an anchoring device; and an alignment target including: a target region having an alignment pattern formed thereon; a socket connected to the target region and configured to fit rotatably to the base, whereby the alignment target is rotatable about the base axis; and an optical indicator for the socket indicating an angle of orientation of the alignment target about the base axis.

In various embodiments of the marker, the base comprises the interface.

In various embodiments of the marker, the interface is coupled to the anchoring device in a manner that aligns the base axis with a base axis of the anchoring device.

In accordance with aspects of the present disclosure, a method for enabling rotation of a marker during surgery without requiring re-registration is provided, the method includes: connecting a base, having a base axis, to an anchoring device; forming an alignment pattern on a target region of an alignment target; connecting a socket to the target region, the socket being at a fixed distance from the target region and being configured to fit rotatably to the base, whereby the alignment target is rotatable about the base axis; providing an optical indicator for the socket indicating an angle of orientation of the alignment target about the base axis; operating an augmented reality system during the surgery on a patient; tracking the alignment target during the surgery; providing a patient tracking vector to the augmented reality system in response to the tracking of the alignment target; calculating a change in the angle of orientation of the alignment target in response to changes in images of the optical indicator; and adding a change-of-orientation vector, based only on the fixed distance and the change in the angle of orientation, to the patient tracking vector so as to update the patient tracking vector.

In various embodiments of the method, the socket is configured to only fit to the base in a plurality of at least two discrete orientations about the base axis.

In various embodiments of the method, the method further includes the socket includes a plurality of apertures equal to the plurality of discrete orientations, and wherein the optical indicator is configured to be visible through one of the apertures indicative of one of the discrete orientations.

In various embodiments of the method, the socket includes a plurality of apertures equal to the plurality of discrete orientations, and wherein the optical indicator is configured to be visible through apertures selected and arranged so as to provide an unambiguous identification of each of the discrete orientations.

In various embodiments of the method, the socket is configured to fit to the base in a plurality of non-discrete orientations about the base axis.

In various embodiments of the method, the socket includes an aperture, and wherein the optical indicator is congruent with the aperture, and wherein a fraction of the optical indicator visible through the aperture is indicative of one of the non-discrete orientations.

In accordance with aspects of the present disclosure, a computer-implemented method for enabling rotation of a marker during surgery without requiring re-registration, wherein the marker is anchored to a patient and configured to fit rotatably to a base, and wherein the marker comprises an alignment pattern and an optical indicator, the optical indicator configured to indicate an angle of orientation of the marker about a base axis of the base and the optical indicator is being at a fixed distance from the alignment pattern, the method including: operating a navigation system during the surgery on the patient; tracking the alignment target during the surgery; providing a patient tracking vector to the navigation system in response to the tracking of the alignment target; calculating a change in the angle of orientation of the alignment target in response to changes in images of the optical indicator; and adding a change-of-orientation vector, based only on the fixed distance and the change in the angle of orientation, to the patient tracking vector so as to update the patient tracking vector.

The present disclosure will be more fully understood from the following detailed description of the embodiments thereof, taken together with the drawings, in which:

### BRIEF DESCRIPTION OF THE DRAWINGS

FIG. 1, which is a schematic illustration of a medical procedure, according to an embodiment of the present invention;

FIG. 2 is a schematic diagram illustrating an exemplary augmented reality assembly used in the procedure, according to an embodiment of the present invention;

FIG. 3 schematically illustrates the situation after an exemplary patient marker has been attached to an exemplary clamp which is inserted and adjusted in a patient, according to an embodiment of the present invention;

FIG. 4A is a schematic perspective view of the marker, and FIGS. 4B-4E are schematic views of different orientations of the marker, according to an embodiment of the present invention;

FIG. 5 is a flowchart describing the use of the marker in the medical procedure, according to an embodiment of the present invention;

FIGS. 6A-6D are schematic views of different orientations of an alternative patient marker, according to an embodiment of the present invention;

FIGS. 7A-7E are schematic views of different orientations of another alternative patient marker, according to an embodiment of the present invention;

FIG. 8 is a flowchart describing the use of the marker of FIGS. 7A-7E, according to an embodiment of the present invention; and

FIG. 9 is a diagram explaining some of the steps of the flowchart of FIG. 8, according to an embodiment of the present invention.

### DETAILED DESCRIPTION OF EMBODIMENTS

### Overview

In an augmented reality system that is used during surgery or a medical procedure on a patient it is necessary to track the position or movement (or both) of the patient. The system typically comprises a head-mounted display worn by a professional performing the surgery, and the tracking, performed e.g., via a tracking system, is required so as to maintain registration of images of the patient that are presented in the display with the professional's view of the patient.

To track the patient's position or movement relative to the display, an alignment target (may be also referred as “target” herein below) may be fixed to the patient and a processor (e.g., via a tracking system) may be configured to track or determine the relative location of the target. In embodiments of the invention the target is fixed to a base of a patient anchoring device such as a clamp or a pin that is clamped or inserted to a bone of the patient, so that the target, when attached to the patient anchoring device, e.g., a clamp base, acts as a patient marker. According to other embodiments of the invention, the patient marker comprises the alignment target coupled to a marker base, wherein the patient marker may be anchored to the patient via an anchoring device, such as a clamp (e.g., a base of the anchoring device). If the surgery is on the spine of the patient, the bone may comprise one or more spinous processes of the patient's vertebrae.

Typically, the tracked position or location of the target of the patient marker is “registered” with or represents the patient (e.g., utilized as a fiducial marker), and this “registration” or representation is used by the processor during the surgery. Tracking or determining of the target location allows maintaining the registration of images of the patient that are presented in the display with the professional's view of the patient and may further allow the display of other tracked elements on the images of the patient, such as tools used during the surgery. The display of the tools may facilitate their navigation by the professional performing the surgery.

However, during the procedure the alignment target may interfere with the surgery being performed, for example, by obstructing the professional's view and/or by restricting the professional's action. In this case, the alignment target may be re-oriented or re-positioned with respect to the anchoring device, e.g., a clamp, and/or the patient, e.g., by re-positioning the target and/or the clamp, to overcome the interference. The re-orientation or re-positioning, may be performed by detaching the target from the clamp, then re-attaching the target to the clamp, or by re-positioning of the clamp itself. The above may necessitate re-registration of the target with the clamp, re-positioning of the clamp with respect to the bone and/or even the performance of an additional incision.

Embodiments of the present invention allow for re-orientation of the target with respect to the patient anchoring device without the necessity of re-registration by the professional and/or the performance of any additional operations. The alignment target comprises a target region having an alignment pattern formed thereon. A socket comprised in the alignment target is fixedly connected at a known distance to the target region. The alignment target and/or the anchoring device base also comprises an optical indicator for the socket indicating an angle of orientation of the alignment target about the base axis. According to some aspects, the socket is configured to fit rotatably to the base of the anchoring device, e.g., a clamp, so that the alignment target is rotatable about a base axis defined by the clamp base. According to some aspects, the patient marker comprises an alignment target coupled to a marker base. The alignment target comprises the optical indicator and configured to rotate around an axis of the marker base.

During the procedure the processor operating the augmented reality system may track the alignment target so as to provide a patient tracking vector to the system, the vector maintaining the registration referred to above. The processor may then calculate a change in the angle of orientation of the alignment target in response to changes in images of the optical indicator. Based only on the change in the angle of orientation and the known target region—socket distance, the processor may calculate a change-of-orientation vector, and then add this vector to the patient tracking vector so as to update the patient tracking vector.

The updated patient tracking vector acts to automatically update the registration of the tracking of the alignment target, indicating the professional's view of the patient, with the patient image data, so that no re-registration and/or any additional operations are necessary.

The terms “position” and “location” may be hereby interchangeably used. Although the description refers to a surgery, the disclosed systems, devices, and methods may be applicable, mutatis mutandis, to any suitable medical procedure. Although the description refers to a spinal surgery or a spinal medical procedure, the disclosed systems, devices, and methods may be applicable, mutatis mutandis, to medical procedures performed with respect to structures or parts of the body other than the spine, including joint trauma related procedures, hip surgery and hip replacement or cranial procedures. Although the description refers to a clamp, the disclosed systems, devices, and methods may be applicable, mutatis mutandis, to any other patient or bone anchoring device, such as a pin inserted into a patient's bone, e.g., into the ilium or a cranial anchoring frame. Although the description refers to an augmented reality system, the disclosed systems, devices and methods may be applicable, mutatis mutandis, to other navigation and/or tracking and display systems, such as stationary tracking and/or display systems, which are not head-mounted, and the like.

### System Description

In the following, all directional references (e.g., upper, lower, upward, downward, left, right, top, bottom, above, below, vertical, and horizontal) are only used for identification purposes to aid the reader's understanding of the present invention, and do not create limitations, particularly as to the position, orientation, or use of embodiments of the invention.

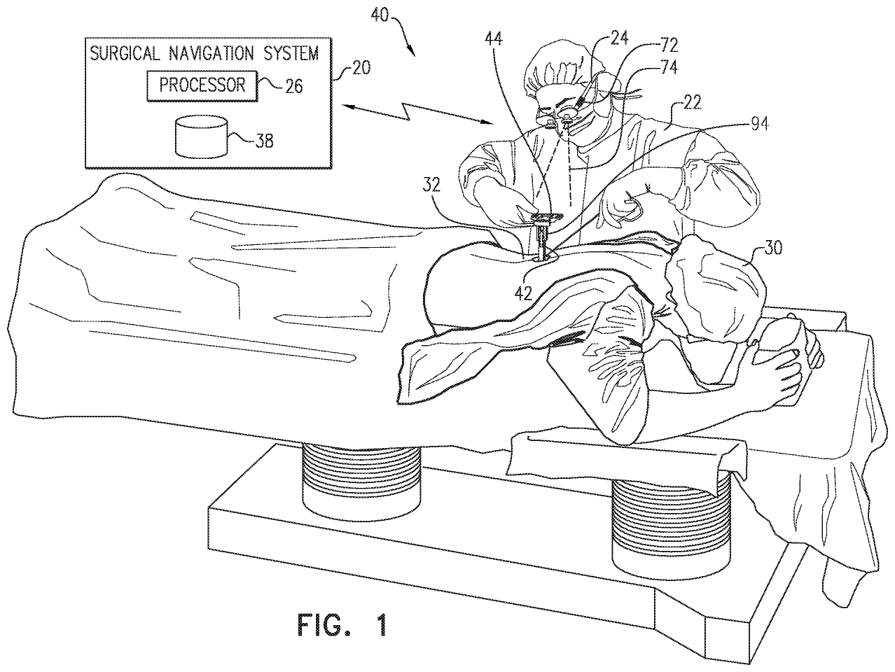

Reference is now made to FIG. 1, which is a schematic illustration of a medical procedure, according to an embodiment of the present invention. During the procedure, performed by a professional 22, the professional uses a surgical navigation system 20, which assists the professional in performance of the procedure. Surgical navigation system 20 comprises a processor 26, which operates elements of the system, and which communicates with an augmented reality assembly 24, worn by professional 22, that is incorporated in the system. While assembly 24 may be incorporated for wearing into a number of different retaining structures on professional 22, in the present description the retaining structure is assumed to be similar to a pair of spectacles. Those having ordinary skill in the augmented reality art will be aware of other possible structures, such as incorporation of the augmented reality assembly into a head-up display that is integrated into a helmet worn by the user of system 20, and all such structures are assumed to be comprised within the scope of the present invention.

In one embodiment processor 26 is assumed to be incorporated within a stand-alone computer, and the processor typically communicates with other elements of the system, including assembly 24, wirelessly, as is illustrated in FIG. 1. Alternatively or additionally, processor 26 may use optical and/or conducting cables for the communication. In further alternative embodiments processor 26 is integrated within assembly 24, or in the mounting of the assembly. Processor 26 is typically able to access a database 38, wherein are stored images, other visual elements and any other type of data, including computer code, used by system 20. Software enabling processor 26 to operate system 20 or assembly 24 (or both) may be downloaded to the processor or to database 38 in electronic form, over a network, for example. Alternatively or additionally, the software may be provided on non-transitory tangible media, such as optical, magnetic, or electronic storage media.

Assembly 24 comprises, inter alia, an image capturing device 72, also termed herein a camera 72, that has a field of view 74. Camera 72 may be configured to capture images in the visible spectrum, non-visible spectrum or both. Assembly 24 and functions of system 20, processor 26, and device 72 are described below. An assembly similar to augmented reality assembly 24, and its operation, are described in U.S. Pat. No. 9,928,629, to Benishti, et al., whose disclosure is incorporated herein by reference.

The medical procedure exemplified here is performed on a patient 30, and during an initial stage of the procedure professional 22 makes an incision 32 into the patient's back. The professional then inserts a spinous process clamp 42, into the incision, so that opposing jaws of the clamp are located on opposite sides of a spinous process. The professional adjusts clamp 42 to grip one or more spinous processes, selected by the professional, of the patient.

The professional attaches an alignment target 44 to a base 94 of the clamp, the target when attached to the base operating as a patient marker 40. Patient marker 40 thus comprises alignment target 44 coupled to base 94. As is described below, patient marker 40 is used by system 20 to determine the position and orientation of patient 30 during the medical procedure.

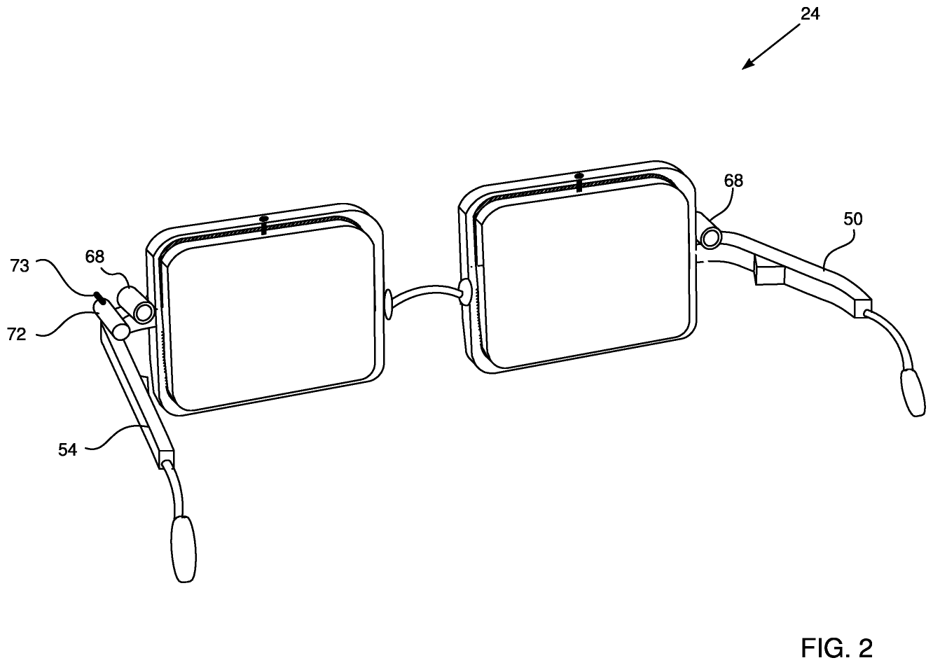

FIG. 2 is a schematic diagram illustrating assembly 24, according to an embodiment of the present invention. As stated above, assembly 24 is configured, by way of example, as a pair of spectacles 50 mounted on a frame 54.

At least one image capturing device 68 or 72 is attached to frame 54. Typically, devices 68 comprise cameras configured to capture images of scenes viewed by the professional's eyes, including images of marker 40 in the visible spectrum.

As stated above assembly 24 comprises camera 72, which is configured to capture images of elements of a scene, including marker 40, in front of assembly 24. The images are produced from radiation projected by a projector 73 that is in the spectrum detected by camera 72. Projector 73 is located in close proximity to camera 72, so that radiation from the projector, that has been retroreflected, is captured by camera 72. The camera typically has a bandpass filter configured to block other radiation, such as that projected by surgical lighting. Typically, camera 72 and projector 73 operate in a non-visible region of the spectrum, such as in the near infra-red spectrum. As is described below, at least some retroreflected radiation is typically received from marker 40, and processor 26 uses the image of the marker produced by camera 72 from the received radiation to track the marker, and thus the position and orientation of patient 30. By tracking the position and orientation of patient 30, the processor is able to present, to professional 22 in assembly 24, images of the patient (e.g., a Computerized Tomography scan) that are correctly registered with the physician's actual view of the patient. According to some aspects, camera 72 may be mounted on frame 54 between the two lenses of spectacles 50. According to some aspects, projector 73 may include two or more projectors. According to another exemplary embodiment, assembly 24 comprises only one capturing device, e.g., camera 72, configured to operate in the non-visible region of the spectrum, such as in the near infra-red spectrum. Camera 72 may be then configured to capture images of scenes viewed by the professional's eyes, including images of marker 40 in the non-visible spectrum. According to some aspects, camera 72 and two projectors 73 may be mounted on frame 54 between the two lenses of spectacles 50. Each of projector 73 may be mounted on each side of camera 72.

Capturing devices 68 and/or 72 and projector 73 may be mounted or positioned on other positions or arranged in other configurations with respect to frame 54 than as illustrated and described with respect to FIGS. 1 and 2, and as may be practiced, implemented or envisioned by persons skilled in the art.

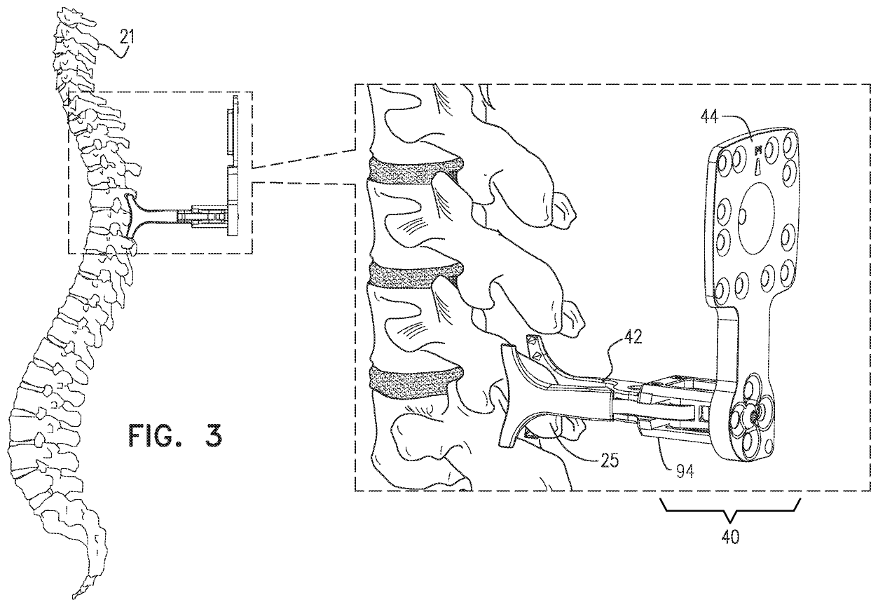

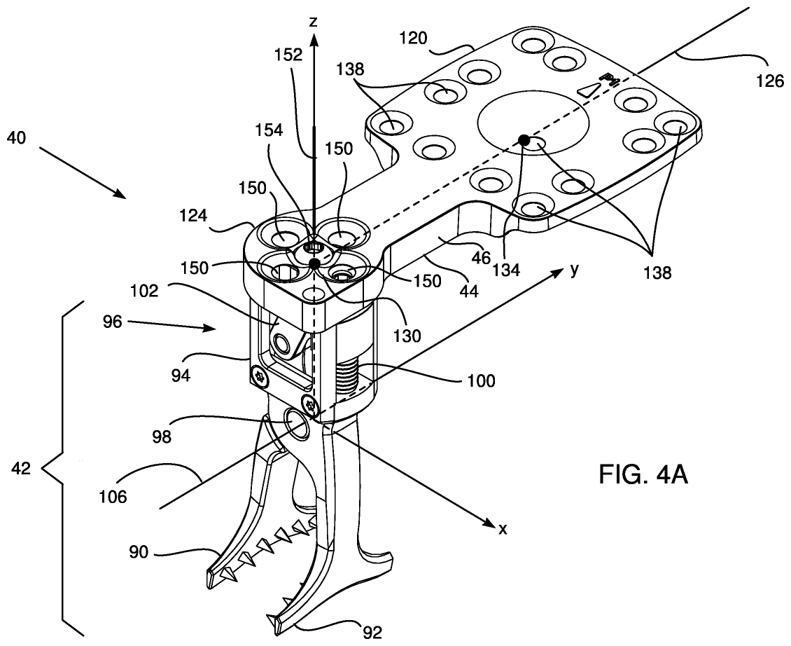

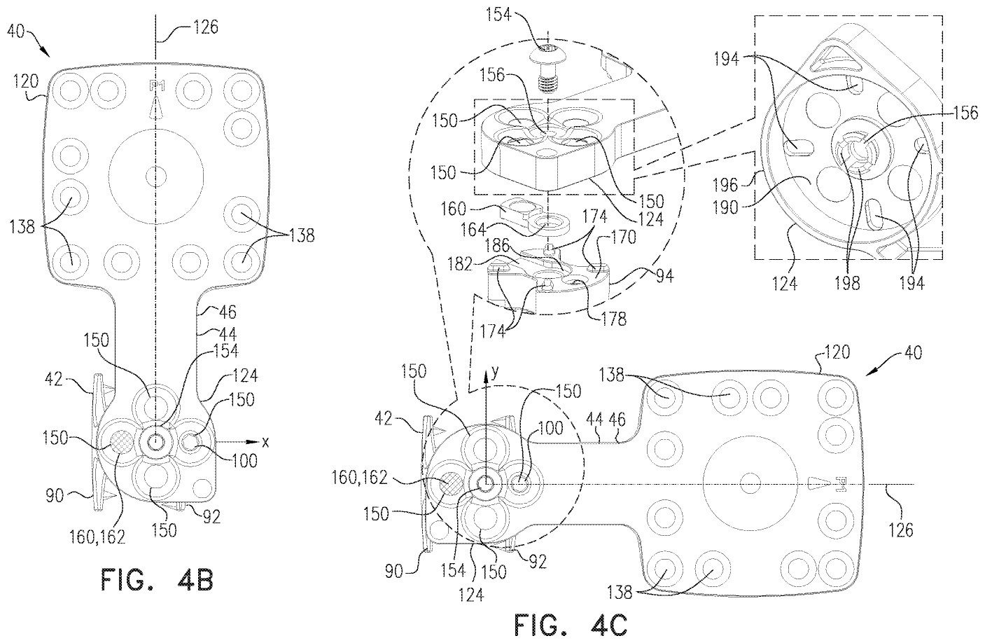

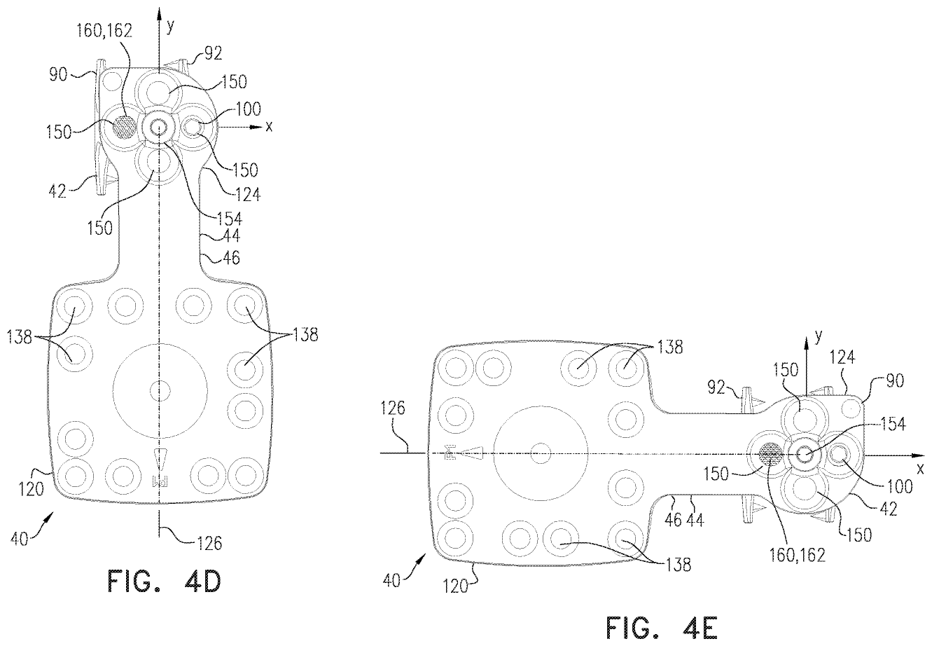

FIG. 3 schematically illustrates the situation after clamp 42 has been inserted and adjusted in patient 30, according to an embodiment of the present invention. Target 44 is then attached to base 94 of the clamp, forming marker 40. The figure illustrates that clamp 42 has been attached to grip a bone 21 of patient 30, specifically to grip a spinous process 25 of vertebrae of the spine of the patient. After attachment, alignment target 44 is external to the patient. As shown in FIG. 3, exemplary clamp 42 comprises teeth, protruding internally from jaws of the clamp, the teeth facilitating the clamp fixedly gripping the spinous processes. An exemplary configuration of marker 40 is described in more detail below with reference to FIGS. 4A-4E.

FIG. 4A is a schematic perspective view of marker 40, and FIGS. 4B-4E are schematic views of different orientations of the marker, according to an embodiment of the present invention. As stated above, marker 40 is formed by attaching alignment target 44 to base 94 of clamp 42. The clamp is described below.

Exemplary clamp 42 comprises a pair of jaws 90, 92 in a lower section of the clamp. The jaws are coupled to clamp base 94 in an upper section of the clamp, the base comprising a jaw adjustment mechanism 96. In the embodiment described herein, jaw 92 is fixed to base 94, and jaw 90 moves with respect to jaw 92, by being rotated about a hinge pin 98. Jaw adjustment mechanism 96 comprises an adjustment screw 100, which is coupled by a lever 102 to jaw 90 so that rotation of the screw causes jaw 90 to approach or retreat from jaw 92. Thus professional 22 is able to cause the jaws of clamp 42 to grip or release a bone, such as spinous process 25, by rotating screw 100. Hinge pin 98 defines a hinge axis 106 about which jaw 90 rotates, and each jaw 90, 92 is substantially parallel to the hinge axis.

For clarity, in the description herein, elements of marker 40 are assumed, by way of example, to be referenced to an xyz set of orthogonal axes, with origin at the center of hinge pin 98. The xyz set of axes is illustrated in FIG. 4A, wherein a y-axis is assumed to correspond to hinge axis 106, an x-axis is orthogonal to a plane including jaws 90 and 92, and a z-axis is orthogonal to the x- and y-axes.

Alignment target 44 comprises a target region 120 and a socket 124, the target region and the socket being fixedly connected together by a connecting rod 46. Alignment target 44, together with its components target region 120 and socket 124, are generally planar, herein termed xy planar since they are assumed to be in a plane parallel to the x- and y-axes. Embodiments of the present invention measure an angle of orientation of alignment target 44 to clamp 42, so that a line 126, constructed from a center 130 of the socket to a center 134 of the target region and extended therefrom, is herein assumed to indicate a direction of orientation of the alignment target 44.

Target region 120, by way of example, is approximately rectangular and comprises optical elements 138. Elements 138 are arranged in a three-dimensional (3D) pattern with no rotational axis of symmetry (other than a trivial axis of symmetry for rotating by 360°), and no mirror plane of symmetry, so that an image of the elements enables an unambiguous determination of the location and orientation of the target region. Elements 138 are typically retroreflectors. According to some aspects, elements 138 are arranges in a two-dimensional (2D) pattern. An entity with an arrangement of optical elements similar to the arrangement herein is described in PCT Patent Application WO2019211741A1, which is incorporated herein by reference.

As stated above, socket 124 is generally planar, and is assumed to define an axis 152 through socket center 130 and orthogonal to an xy plane. In cases where socket center 130 lies on the z-axis, axis 152 is coincident with the z-axis, as is illustrated in FIG. 4A. Socket 124 comprises four substantially similar apertures 150 which are distributed symmetrically about axis 152. Socket 124 comprises a central hole 156, and a screw 154 is configured to penetrate the hole and connect the socket to an upper surface 170 of clamp base 94, as is illustrated in the call-out of FIG. 4C. Once connected, a base axis, comprising an axis orthogonal to surface 170 through central hole 156, corresponds to axis 152.

Surface 170 is xy planar, being parallel to an xy plane, and comprises four protrusions 174 distributed symmetrically about the z-axis. There is an aperture 178 in the surface providing access to adjustment screw 100, and the positions of apertures 150 are selected so that regardless of the orientation of target 44, access to screw 100 is available through one of apertures 150. There is also a shaped indentation 182, comprising an are 186, in surface 170, the indentation being shaped to accept a colored or retroreflective insert 160.

As is also illustrated in FIG. 4C, socket 124 comprises a planar lower surface 190, within which are inset four indents 194, distributed symmetrically about socket central hole 156, and configured to mate with protrusions 174. Surface 190 is surrounded by a circular wall 196. Extending from surface 190 are a plurality of arcs 198, also distributed symmetrically about socket central hole 156, and configured to mate with arc 186 of indentation 182.

FIGS. 4B-4E illustrate the four different discrete orientations that exemplary alignment target 44 is able to make with clamp 42, when the target is connected to the clamp with screw 154 so that socket 124 mates with base 94. Assuming that the positive y-axis of the clamp corresponds to an orientation of 0°, and those orientations are measured as clockwise rotations about the z-axis from the y-axis, FIGS. 4B, 4C, 4D, and 4E correspond respectively to the target having discrete orientations of 0°, 90°, 180°, and 270°. At each orientation arcs 198 mate with arc 186, protrusions 174 mate with indents 194, and wall 196 mates with outer circular edges of base 94, the mating ensuring that socket 124 is centered with respect to the z-axis.

As is illustrated in FIGS. 4B-4E, at each orientation insert 160 is visible through one of apertures 150, and the visible insert acts as an optical indicator 162 of the orientation. During operation of system 20, processor 26 calculates coordinates of a directed line segment between indicator 162 and center 134, the coordinates acting as an orientation metric. For each orientation there is a unique directed line segment, i.e., a unique orientation metric, so that processor 26 is able to use the calculated coordinates as an orientation indicator.

Table I below shows coordinates, the orientation metric, of the directed line segment for each of the exemplary four orientations of target 44. (For clarity, Table I, and Table II below, are drawn to a two-dimensional system, and may be adapted, mutatis mutandis, to a three-dimensional or higher system.) The coordinates are calculated assuming that indicator 162 lies on a circle radius r centered at center 130 of socket 120, and that there is a separation of D between center 130 and center 134.

TABLE I

| | Orientation Indicator |
|---|---|
| Orientation | Orientation Metric |
| 0° | (−r, 0) − (0, D) = (−r, −D) |
| 90° | (−r, 0) − (D, 0) = (−r − D, 0) |
| 180° | (−r, 0) − (0, −D) = (−r, D) |
| 270° | (−r, 0) − (−D, 0) = (D − r, 0) |

As is described herein, marker 40 is used to track the location of patient 30, typically the patient's bone, with respect to assembly 24, by tracking the location of target region 120. Since the location of the target region is fixed with respect to the patient's bone to which marker 40 is clamped, because the marker is inflexible, tracking of the patient's bone may be accomplished by tracking of the target region, and adding a fixed adjustment vector due to the differing physical positions of the target region and the bone.

Furthermore, since the target region positions have a one-to-one correlation with the orientations, and since the different target region positions are in known geometric relations to each other, these geometric relations may be pre-programmed as change-of-orientation vectors and used to continue tracking the patient when the target region orientation is changed.

For example, if target region 120 is a distance D from socket 124, then the target region positions for the orientations 0°, 90°, 180°, and 270° (illustrated in FIGS. 4B, 4C, 4D, and 4E) may be respectively represented by the two-dimensional ordered pairs (0, D), (D, 0), (0, -D), and (-D, 0). If an initial target region is in the 0° orientation, then the geometric relations, i.e., the change-of-orientation vectors, to the other three orientations are as given in Table II:

TABLE II

| New | Change-of-orientation |
|---|---|
| Orientation | Vector (From 0° Orientation) |
| 90° | (D, 0) − (0, D) = (D, −D) |
| 180° | (0, −D) − (0, D) = (0, −2D) |
| 270° | (−D, 0) − (0, D) = (−D, −D) |

It will be understood that the three change-of-orientation vectors presented in Table II do not vary with movement of marker 40. The vectors depend only on the initial and final orientation of the target region, and so, as stated above, may be pre-programmed. It will also be understood that sets of three change-of-orientation vectors from the other possible initial orientations (90°, 180°, and) 270° may be calculated as for Table II, and may also be pre-programmed.

As is explained further below, in embodiments of the present invention, when the target region orientation changes a processor adds an appropriate change-of-orientation vector to an initial tracking vector of a patient marker. This enables continuous tracking of a patient by the marker without re-registration of the marker.

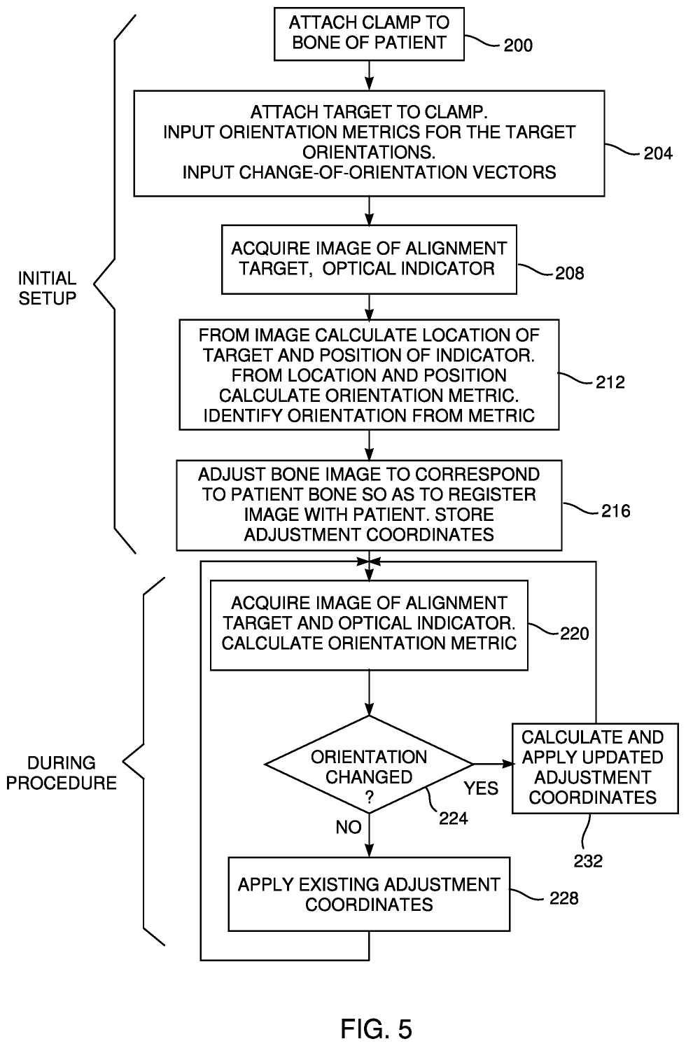

FIG. 5 is a flowchart describing the use of a marker such as marker 40 in the medical procedure referred to above, according to an embodiment of the present invention.

In an initial step 200, professional 22 attaches clamp 42 or any other patient anchoring device to a bone of patient 30, herein assumed to comprise spinous process 25 of the patient, by rotating screw 100.

In a target attachment step 204, the professional attaches an alignment target to the patient anchoring device in a specific orientation. The alignment target includes an optical indicator indicating the marker current orientation. For example, alignment target 44 may be attached to the clamp, by aligning socket 124 with surface 170 of clamp base 94, and screwing screw 154. It will be understood that the attached orientation is one of the four orientations illustrated in FIGS. 4B-4E, so that after the attachment, insert 160 is visible through one of apertures 150, and acts as optical indicator 162.

The values of r and D, and the sets of orientation metrics for the orientations of the target, described above with reference to Table I, may be input to or accessed by processor 26. In addition, the sets of change-of-orientation vectors coordinates, described above with reference to Table II, may also be input to or accessed by the processor.

In an image acquisition step 208, an image of optical elements and of the optical indicator of the alignment marker is captured via the head mounted assembly. With reference to FIGS. 1 and 2, an image of optical elements 138 of target region 120 and of indicator 162, is captured, e.g., using camera 72 and/or one or more devices 68. Optionally the image may include the bone of patient 30, or of a region close to the bone, such as incision 32 and/or a tool navigated in a region close to the bone or alignment target 44. The tool may be mounted with or include a tool marker. The tool location may be then identified with respect to the patient via the tool marker and the alignment target (the patient marker).

In an analysis step 212, the processor analyzes the acquired image to identify the alignment target and/or to find a location, comprising an orientation and a position, of the alignment target.

For example, the processor analyzes the acquired image to identify and/or find a location, comprising an orientation and a position, of target region 120. The position of target region is herein, by way of example, assumed to correspond to center 134. Once the target region location has been found, the processor may initiate tracking of the target region, for example, by capturing images of the target region, e.g., once in a predefined time interval.

The processor also identifies the optical indicator of the alignment target in the acquired image. For example, processor 26 may find a position of indicator 162 in the image. From coordinates of the two positions the processor calculates coordinates of an orientation metric (as in Table I) joining the positions, and from the metrics stored in step 204, identifies the orientation of step 204.

In an adjustment step 216, the processor utilizes the image, e.g., of the bone, of the region close to the bone, and/or of a tool navigated in the region close to the bone, acquired in step 208, to generate an augmented reality display to professional 22 in augmented reality assembly 24. The professional adjusts coordinates of the presented image to correspond with the actual image visible to the professional through the assembly, and the processor stores the adjustment coordinates. The processor then applies the stored adjustment coordinates as an adjustment vector, together with the tracking of the tracked region (initiated in step 212), to the presented image, so as to register the presented image with patient 30 on a continuing basis.

The processor continues to apply the stored adjustment vector, which acts as a patient tracking vector, as long as the orientation of step 204 is unchanged. It will be understood that the registration using the stored adjustment vector counteracts any relative movement between the patient and assembly 24.

Steps 200-216 correspond to an initial setup of navigation system 20. Steps 220-232, described below, correspond to steps of the flowchart that may be implemented during the medical procedure for which system 20 is used.

In a continuing imaging step 220, an image of the alignment target and optionally of additional elements of interest (e.g., tools used during the procedure or of a region of interest on the patient's body) is continuously acquired (e.g., once in a predefined time interval). For example, an image of target 44, including target region 120 and indicator 162 is continuously acquired via assembly 24 (optionally controlled by processor 26 but not necessarily). From the images of target region 120 and indicator 162, the processor calculates an orientation metric.

In a decision step 224 the processor checks if the metric calculated in step 220 is different from that calculated in step 212, so as to check if the target region has changed orientation. If the decision returns negative, i.e., there is no change in orientation, then in a continuation step 228 the processor continues to use the existing adjustment coordinates, i.e., those of step 216.

If the decision returns positive, i.e., there is a change in orientation, then in an update step 232 the processor calculates updated adjustment coordinates, by adding the appropriate change-of-orientation vector, from step 204, to the existing adjustment coordinates. The processor applies the updated coordinates in presenting the image of the patient, e.g., of the region close to the bone, to professional 22 in augmented reality assembly 24.

It will be understood that a positive return of decision 224 is typically caused by professional 22 changing the orientation of the alignment target. The professional may change the orientation for example by unscrewing screw 154, realigning socket 124 on surface 170 so that target 44 is in a new orientation, then screwing screw 154 to fix the target in its new orientation. In some embodiments the professional may pause the tracking of target 44 while positioning target 44 in its new orientation.

The professional typically repositions target 44 to improve access to, and/or to improve visibility to, part of the patient. It will be understood that there is no need to repeat steps 200-216 after the repositioning, since the new adjustment coordinates can be calculated from the known geometric relations of the two orientations of target 44. (It will also be understood that regardless of any repositioning, adjustment screw 100 is always accessible via one of apertures 150, since the screw aligns with the apertures.)

From steps 228 and 232 control returns to decision step 220, so that during the procedure the processor applies steps 220-232 iteratively.

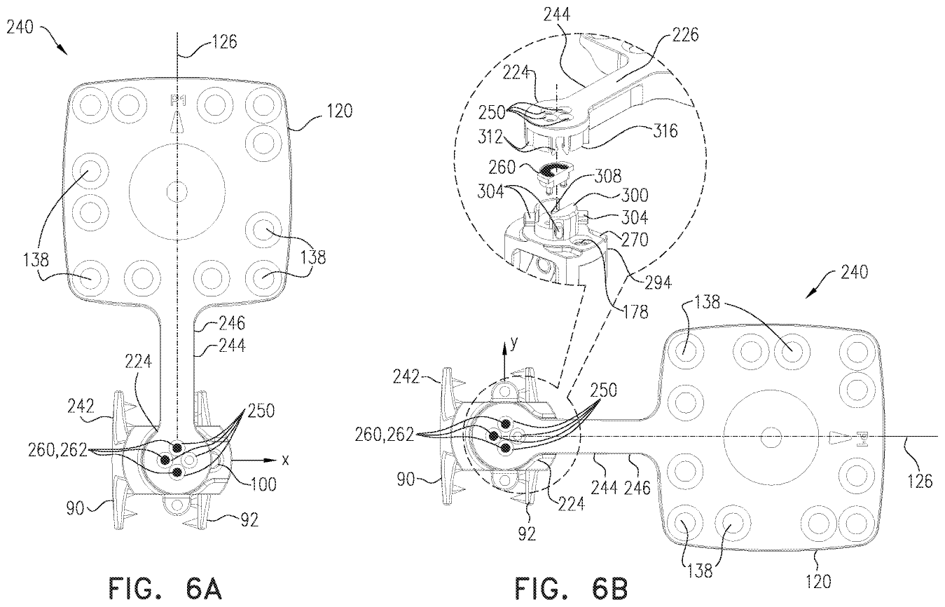

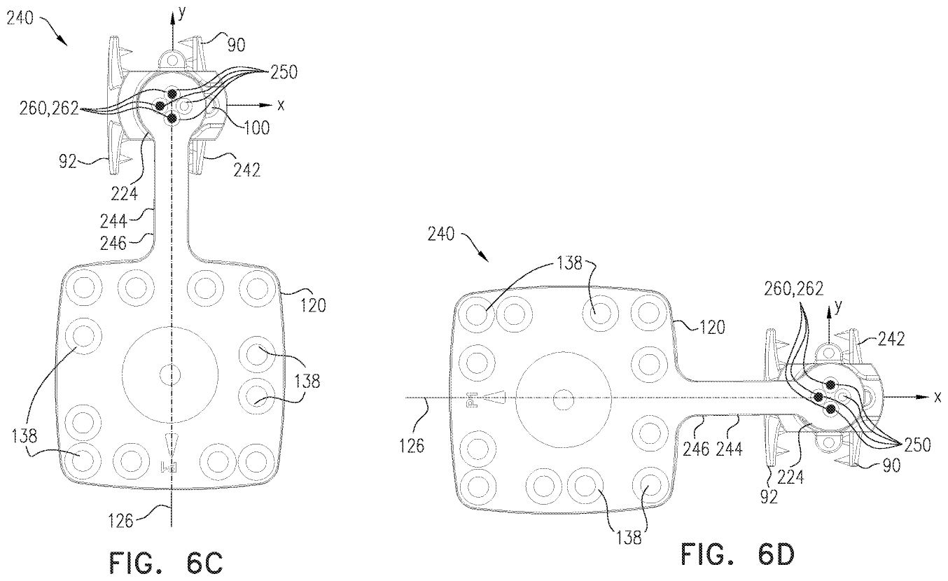

FIGS. 6A-6D are schematic views of different orientations of a patient marker 240, according to an embodiment of the present invention. Marker 240 comprises a clamp base 294 which is coupled to an alignment target 244. Clamp base 294 is the base of a clamp 242. Apart from the differences described below, the operation of marker 240, clamp 242, base 294, and target 244 is generally similar to that of marker 40, clamp 42, base 94, and target 44, and elements indicated by the same reference numerals in both markers are generally similar in construction and in operation. As for marker 40, target 244 has four different discrete orientations with respect to clamp 242. The axes for marker 240 are as for marker 40.

In contrast to upper surface 170 of clamp base 94 of clamp 42, an upper xy surface 270 of clamp base 294 of clamp 242 is surmounted by a circular turret 300. Fixed to turret 300 are four protuberances 304, distributed symmetrically about a center of the turret, and lying in an xy plane, parallel to surface 270. Turret 300 comprises a recess 308, which is configured to accept an insert 260 that typically is colored or retroreflective.

As for socket 124, a socket 224 of target 244 comprises four apertures 250 distributed symmetrically about a center of the socket. However, socket 224 is smaller than socket 124, and regardless of target orientation, an aperture 250 does not align with screw 100. Rather, socket 224 is small enough so that in at least some target orientations, e.g. those of FIG. 6A, 6C and 6D, screw 100 is accessible from outside the socket.

A connecting rod 246 connects target 120 to socket 224, but typically has a smaller width than rod 46.

Socket 224 has a lower circular wall 316 which has an internal surface which is configured to mate with turret 300. Set within the wall are four clips 312 which are distributed symmetrically about a center of the socket, and which are positioned to mate with protuberances 304. When clips 312 mate with protuberances 304, they hold socket 224 so that wall 316 surrounds and contacts an outer surface of turret 300, and so the socket is fixed to the turret.

Because protuberances 304 and mating clips 312 are distributed symmetrically, it will be understood that target 244 can mate with clamp 242 in one of four orientations illustrated in FIGS. 6A-6D, and that in each orientation a center of socket 224 aligns with a center of turret 300.

Unlike marker 40, where optical indicator 162 is formed from the visibility of insert 160 through one of apertures 150, in marker 240 an optical indicator 262 comprises insert 260 as viewed through three apertures 250, as illustrated in FIGS. 6A-6D.

The description of the flowchart of FIG. 5 applies to marker 240, mutatis mutandis. For example, a position of indicator 262 may be assumed to be the position of the central one of the three apertures 260.

Markers 40 and 240 each have four symmetrically distributed discrete orientations. However, embodiments of the present invention may have other numbers of symmetrically distributed orientations, where the number may be as little as two.

The number of apertures 150 corresponds to the number of discrete orientations. As exemplified by indicator 162 the number of apertures used to generate the optical indicator may be a single aperture. Alternatively, as exemplified by indicator 262, the number of apertures used to generate the optical indicator may comprise any fixed number of apertures that is at least one less than the total number of apertures. In this case the apertures are selected and arranged so that when rotated, they provide an unambiguous identification of each of the discrete orientations.

Thus, for four apertures, corresponding to four discrete orientations, the indicator may be two adjacent apertures, but not two apertures opposite each other, since two apertures opposite each do not provide an unambiguous identification of each orientation.

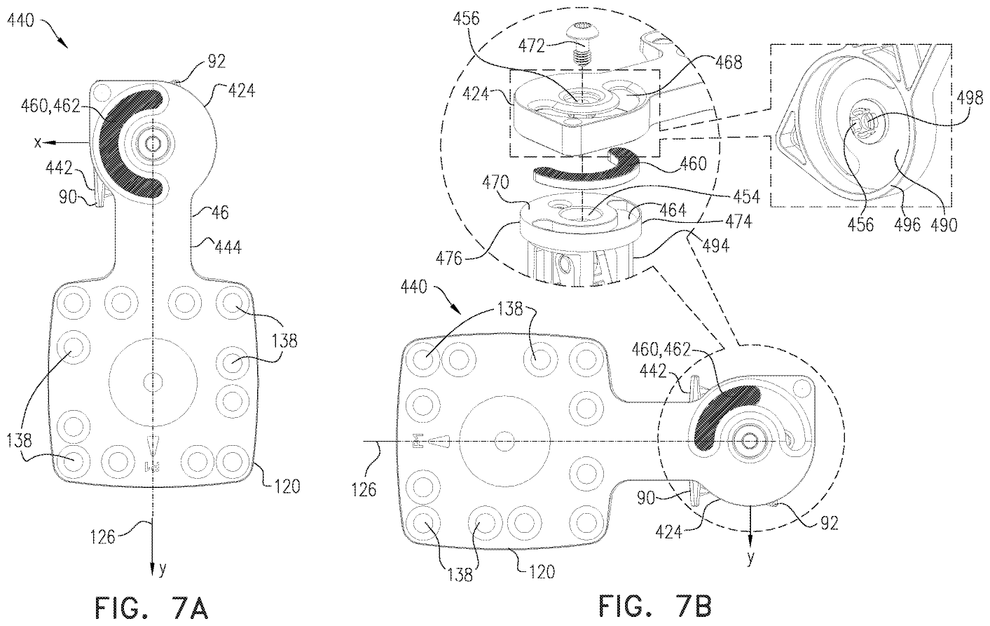

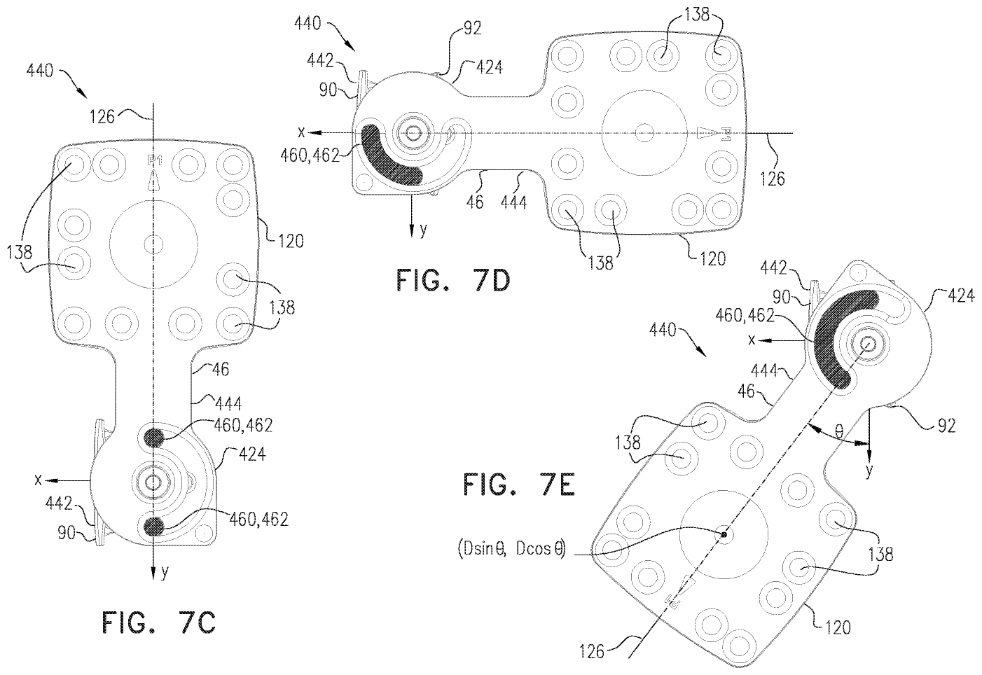

FIGS. 7A-7E are schematic views of different orientations of a patient marker 440, according to an embodiment of the present invention. Marker 440 comprises a clamp base 494 which is coupled to an alignment target 444. Clamp base 494 is the base of a clamp 242. Apart from the differences described below, the operation of marker 440, clamp 442, base 494, and target 444 is generally similar to that of marker 40, clamp 42, base 94, and target 44, and elements indicated by the same reference numerals in both markers are generally similar in construction and in operation. Unlike marker 40, where target 44 can only make discrete orientations with respect to clamp 42, target 444 in marker 440 can make multiple non-discrete, substantially continuous, orientations varying from 0°-360° with respect to clamp 442.

FIGS. 7A-7E have been drawn on the same set of xyz axes as for marker 40 (although the axes are rotated 180° compared to those of FIGS. 4B-4E), and orientations are measured as clockwise rotations about the z-axis from the y-axis. FIGS. 7A, 7B, 7C, and 7D, correspond respectively to the target having orientations of 0°, 90°, 180°, and 270° relative to the clamp.

FIG. 7E illustrates the target having an orientation of θ relative to the clamp, where 0°≤θ<360°, and coordinates of a center point of the target region have been marked as (Dsinθ, Dcosθ) where D is the distance of the target region center point from the z-axis.

In contrast to upper surface 170 of clamp base 94 of clamp 42, an upper xy plane surface 470 of an upper plate 476 of clamp base 494 is circular. Surface 470 has a central circular indent 454 symmetrically situated in the surface, and the indent is terminated at its lower end by a female thread. Surface 470 also has an indent 464 which is in the form of a semicircular arc, centered on a center of circular surface 470. An insert 460 that is a semicircular arc and that is typically colored or retroreflective is inserted into indent 464, and the insert is dimensioned so that an upper surface of the insert is level with surface 470.

A socket 424 of target 444 comprises a planar lower surface 490 which is surrounded by a circular wall 496 that is configured to mate with an outer cylindrical surface 474 of plate 476. Extending from surface 490 are a plurality of arcs 498, distributed symmetrically about a socket central hole 456, configured to mate with indent 454. Socket 424 also comprises a semicircular aperture 468, which is congruent to insert 460.

Target 444 is coupled to clamp 442 by fitting socket 424 to plate 476 so that wall 496 mates with surface 474, and so that arcs 498 mate with indent 454. Once so coupled, target 444 may be held fixedly in place in any orientation selected by professional 22, by screwing a screw 472 into the female thread terminating indent 454.

During a procedure processor 26 is able to determine the orientation of the target, as a value between 0° and 360°, by the imaging of insert 460, and the use of the imaged insert as an optical indicator 462 of the orientation. In one embodiment processor 26 determines the orientation by finding the fraction of the insert visible through aperture 468, as well as a location of the visible insert.

In embodiments of the invention, the fraction may comprise a fractional area of the insert, or alternatively or additionally, a fractional linear dimension, such as an arc length, of the insert. In some embodiments the fractional linear dimension may be measured using a Vernier scale.

Thus, FIG. 7A illustrates a maximum of the insert visible through the aperture, corresponding to an orientation of 0°, and FIG. 7C illustrates a minimum of the insert visible through the aperture, corresponding to an orientation of 180°. FIG. 7B, corresponding to an orientation of 90° illustrates half of the insert visible, the visible half being located below the x-axis, and FIG. 7D, corresponding to an orientation of 270°, illustrates half of the insert visible, the visible half being located above the x-axis.

Other methods for determining the orientation of the target from the imaged insert, such as by finding coordinates of the endpoints of the imaged insert as well as coordinates of an intermediate point on the image, will be apparent, and all such methods are assumed to be comprised within the scope of the present invention.

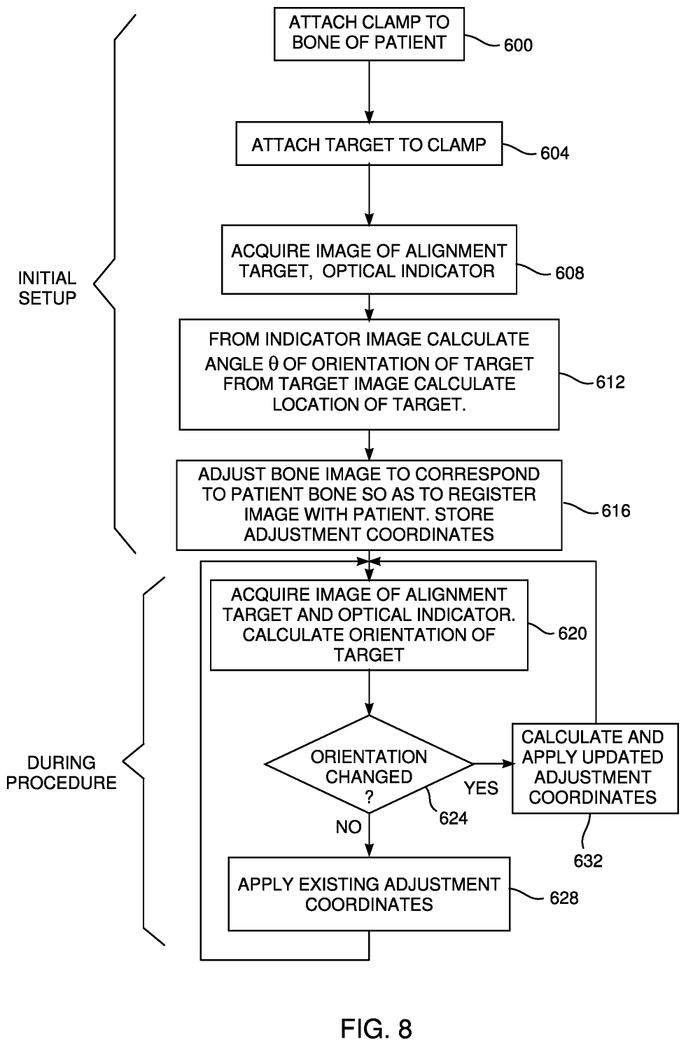

During a procedure, processor 26 determines the orientation of target 444 from imaging optical indicator 462, as described above. The flowchart of FIG. 8 below describes how the processor uses the values of the orientation during the procedure.

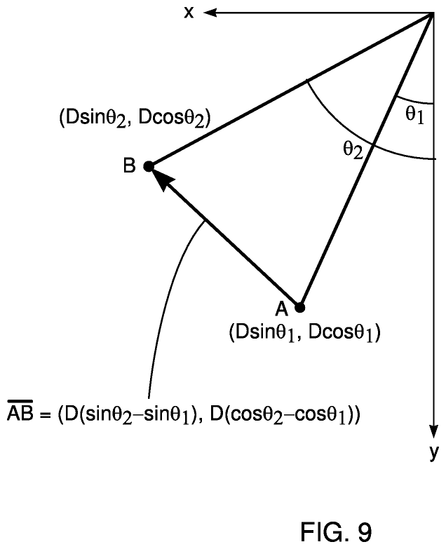

FIG. 8 is a flowchart describing the use of a marker such as marker 440 in the medical procedure referred to above, and FIG. 9 is a diagram explaining some of the steps of the flowchart, according to an embodiment of the present invention.

An initial step 600, wherein clamp 442 is attached to the patient's bone, is substantially similar to initial step 200, described above.

In an attachment step 604, professional 22 attaches socket 424 to base 494, in any convenient orientation of target 444 to clamp 442. The professional uses screw 472 to fix the target to the clamp base.

An imaging step 608 is substantially similar to step 208, described above.

In an analysis step 612, the processor analyzes the image of indicator 462, as described above with reference to FIG. 7E, to determine an angle of orientation θ of the target with clamp 42. In addition, the processor calculates coordinates of the location of target region 120 from the image of the region acquired in step 608.

An adjustment or registration step 616 is substantially as described above for step 216. Thus, in step 616, coordinates of a presented image are adjusted or registered to correspond with an actual image visible to the professional through augmented reality assembly 24. Processor 26 stores the adjustment coordinates, and applies the stored coordinates as an adjustment vector so as to register and track the presented image with patient 30.

Steps 600-616 correspond to an initial setup of navigation system 20 for marker 440. Steps 620-632, described below, correspond to steps of the flowchart that may be implemented during the medical procedure for which system 20 is used.

In a continuing imaging step 620, an image of target 444, including target region 120 and indicator 462 is acquired, e.g., once in a predefined time interval. Optionally, an image of patient 30 may be also acquired. From the image of indicator 462, the processor calculates an angle θ of the orientation of target region 120 relative to the clamp.

In a decision step 624 the processor checks if the angle calculated in step 620 is different from that calculated in step 612, so as to check if the target region has changed orientation. If the decision returns negative, i.e., there is no change in orientation, then in a continuation step 628 the processor continues to use the existing adjustment coordinates, i.e., those of step 616, as an adjustment vector.

If decision 624 returns positive, i.e., there is a change of orientation, then in an update step 632 the processor calculates a change-of-orientation vector, to be added to the existing adjustment vector, so as to enable the processor to maintain registration of images of patient 30 with the patient.

FIG. 9 is a schematic diagram illustrating how the processor calculates the change-of-orientation vector, according to an embodiment of the present invention. A line segment PA, having a length D, represents an initial orientation of adjustment target 444, where A is the center of region 120 and θ₁ is the orientation of the center, both values being measured in step 612. A has coordinates (Dsinθ₁, Dcosθ₁).

A line segment PB, having a length D, represents a subsequent orientation of adjustment target 444, where B is the center of region 120 and θ₂ is the orientation of the center, both values being measured in step 620. B has coordinates (Dsinθ₂, Dcosθ₂).

Processor 26 calculates a change-of-orientation vector [AB] as the difference between the coordinates of B and the coordinates of A, as in equation (1):

$$[ AB ] = ( D ( sin {θ}_{2} - sin {θ}_{1} ) , D ( cos {θ}_{2} - cos {θ}_{1} ) ) \qquad ( 1 )$$

Returning to the flowchart of FIG. 8, in step 632 the processor adds a change-of-orientation vector, calculated as described for equation (1), to the existing adjustment vector.

From steps 628 and 632 control returns to imaging step 620 and following that decision step 624, so that during the procedure the processor applies steps 620-632 iteratively.

A positive return of decision 624 is typically caused by professional 22 changing the orientation of target 444 by loosening then tightening screw 472. In some embodiments the professional may pause the tracking of target 444 while positioning the target in a new orientation. It will be understood that there is no need to repeat steps 600-616 after any re-orientation, since by iterating steps 620-632 the processor continues to correctly register any acquired image of patient 30 with the patient.

According to some aspects, a patient marker comprising an alignment target and a marker base, which may be anchored to the patient via an anchoring device, is herein disclosed. The alignment target is coupled to the marker base. The alignment target is configured to rotate around an axis of the marker base. The alignment target comprises an optical indicator, The optical indicator indicates the angle of orientation of the alignment target about the marker base axis. The patient marker further comprises an anchoring interface. The anchoring interface is coupled to the marker base. According to some aspects, the marker base comprises the anchoring interface. The alignment target may comprise a socket configured to fit rotatably to the target base. The patient marker is configured to be mounted on a patient anchoring device, e.g., on a base of the anchoring device, via the anchoring interface in a fixed manner and such that the marker base axis is aligned with the anchoring device base axis (e.g., a clamp base axis). The optical indicator may be, for example, in the form of and may be configured to operate according to optical indicators 162, 262 or 462, as described in and with respect to FIGS. 4A-4E, 6A-6D and 7A-7E correspondingly. The alignment target may be configured to rotate around an axis of the marker base, for example according to the rotation mechanisms describe in and with respect to FIGS. 4A-4E, 6A-6D and 7A-7E. Persons skilled in the art will understand how to implement different mechanisms for the optical indicator and different rotation mechanisms for the alignment target, including those disclosed herein, in a patient marker comprising a rotatable alignment target coupled to a base of the marker. The description above explains how, for example, an optical indicator such as indicator 162, together with the optical elements 138 of alignment target 44, may be used to find an angle of orientation of the alignment target about an axis of a base to which the alignment target is connected. The base may be a marker base or an anchoring device base, such as a clamp base. It will be understood that optical elements 138 identify the alignment target.

In some embodiments of the present invention, professional 22 uses a surgical tool in the medical procedure referred to above (FIG. 1). The tool has a tool marker connected to the tool or integrated with the tool in a predefined location, and the augmented reality assembly (e.g., augmented reality assembly 24) is configured to enable the tracking (e.g., by processor 26) of the position of the tool tip. In these embodiments, instead of using an optical indicator such as optical indicator 162 to identify the angle of orientation of the alignment target, professional 22 may use the tool to find the angle, as is described below.

The patient anchoring device or the marker base may comprise a fixed tool point (also will be referred as “tool point” herein below). The tool point location is configured to be fixed with respect to the anchoring device and thus with respect to the patient. For example, the fixed tool point may be connected to a base of the anchoring device. In general, the tool having the tool marker mounted thereon is calibrated prior to its use in a medical procedure via the augmented reality assembly. In the calibration process, a spatial ratio is determined between the tool marker and the tip of the tool, such as a vector between the tool marker (e.g., a retroreflective predefined point on the tool marker) and the tool tip. The professional may touch the fixed tool point with the tip of the calibrated tool, while the alignment target of the patient marker is oriented in a specific orientation with respect to the anchoring device. An image of the tool marker and the oriented patient marker is captured by the capturing device of the augmented reality assembly. A location of the fixed tool point with respect to the alignment target is determined by a processor, such as processor 26, based on the image. Following that, the orientation of the alignment target with respect to the anchoring device and thus the patient may be determined. According to some aspects, the professional may be directed by the augmented reality assembly (e.g., by a software executed by processor 26) to touch the fixed tool point with the tip of the tool. According to some aspects, the alignment target has a plurality of predefined orientation and a vector between the alignment target and the fixed tool point in each orientation is calculated in advance. In such a case, a spatial area corresponding to each predefined orientation of the target alignment may be determined and used in determining the orientation of the alignment target, e.g., instead of an accurate calculation of the alignment target location. As described above, the capturing device continuously capture images of the area of interest, including the patient marker and the tool marker. For each image captured via the capturing device, the processor may check if the tool tip is substantially located at a possible tool point location, corresponding to a possible alignment target orientation, to identify the touching of the tool point by the tool tip. Identification of a specific such location in one or more subsequent images may determine the orientation of the alignment marker.

Those having ordinary skill in the art will be able to adapt the description, mutatis mutandis, for various configurations of alignment targets, including targets 44, 244 and 444.

It will also be understood embodiments of the invention may measure discrete changes of orientation e.g., of 0°, 90°, 180°, or 270°, or continuous changes of orientation through substantially any angle between 0° and 360°.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1.-22. (canceled)

23. A marker for image guided surgery, the marker comprising:
a base configured to be attached to a patient;
an alignment target extending from a first end to a second end, the first end being configured to fit rotatably to the base so that the alignment target is rotatable about an axis with respect to the base,
wherein the second end of the alignment target comprises a target region having an alignment pattern formed thereon; and
an optical indicator configured to be visible through at least one aperture of the first end of the alignment target to indicate an angle of orientation of the alignment target about the axis.

24. The marker of claim 23, wherein the optical indicator is positioned within an indentation of the base.

25. The marker of claim 23, wherein the optical indicator is positioned between the first end of the alignment target and the base.

26. The marker of claim 23, wherein the at least one aperture comprises a plurality of apertures.

27. The marker of claim 26, wherein the optical indicator is configured to be visible through no more than one of the plurality of apertures at a time.

28. The marker of claim 23, wherein the at least one aperture comprises a semicircular arc.

29. The marker of claim 23, wherein the optical indicator is congruent with the at least one aperture, and wherein a fraction of the optical indicator visible through the at least one aperture is indicative of the angle of orientation of the alignment target about the axis.

30. The marker of claim 23, wherein the first end of the alignment target is configured to only fit to the base in a plurality of discrete orientations about the axis with respect to the base.

31. The marker of claim 23, wherein the alignment target comprises a connecting rod that connects the first end to the second end.

32. The marker of claim 23, wherein the first end of the alignment target comprises a socket configured to fit rotatably to the base.

33. The marker of claim 23, wherein the base is configured to be attached to the patient by a clamp that is configured to be attached to a bone of the patient.

34. The marker of claim 23, wherein the base is configured to be attached to the patient by a pin that is configured to be attached to a bone of the patient.

35. A marker for image guided surgery, the marker comprising:
a base configured to be attached to a patient;
an alignment target extending from a first end to a second end, the first end being configured to fit to the base in a plurality of discrete orientations about an axis with respect to the base,
wherein the second end of the alignment target comprises a target region having an alignment pattern formed thereon; and
an optical indicator configured to be visible through at least one aperture of the first end of the alignment target to indicate an angle of orientation of the alignment target about the axis.

36. The marker of claim 35, wherein the at least one aperture comprises a plurality of apertures.

37. The marker of claim 36, wherein the optical indicator is configured to be visible through no more than one of the plurality of apertures at a time.

38. The marker of claim 35, wherein the at least one aperture comprises a semicircular arc.

39. The marker of claim 35, wherein the optical indicator is congruent with the at least one aperture, and wherein a fraction of the optical indicator visible through the at least one aperture is indicative of the angle of orientation of the alignment target about the axis.

40. The marker of claim 35, wherein the first end of the alignment target comprises a socket configured to fit to the base in the plurality of discrete orientations.

41. The marker of claim 35, wherein the base is configured to be attached to the patient by a clamp that is configured to be attached to a bone of the patient.

42. The marker of claim 35, wherein the base is configured to be attached to the patient by a pin that is configured to be attached to a bone of the patient.
